(19) United States
(12) Patent Application Publication
(10) Pub. No.: US 2006/0008434 A1
(43) Pub. Date: Jan. 12, 2006

(54) Deodorant body wash with lotion

(76) Inventors: Michael A. Knopf, Randolph, NJ (US); Michele Polk, Flanders, NJ (US); Frank A. Lucia III, Wantage, NJ (US); William C. Wohland, Succasunna, NJ (US); Ralph Macchio, Sparta, NJ (US)

Correspondence Address: Schwegman, Lundberg, Woessner & Kluth, P.A., P.O. Box 2938, Minneapolis, MN, 55402, US

(21) Appl. No.: 11/102,583

(22) Filed: Apr. 8, 2005

Related U.S. Application Data
(63) Continuation-in-part of application No. 10/710,052, filed on Jun. 15, 2004.
(60) Provisional application No. 60/521,565, filed on May 25, 2004.

(51) Int. Cl. A61K 8/28

(52) U.S. Cl. 424/65

(57) ABSTRACT

Embodiments of the invention described herein include a formulation comprising: a deodorant, a structurant and an emulsion comprising a homogenized mixture of wax and alcohol components, at least one of which is a surfactant, wherein the formulation comprises a stable lamellar or spherulite phase.

### RELATED APPLICATION

This application is a continuation-in-part of U.S. patent application Ser. No. 10/710,052, filed Jun. 15, 2004, which claims priority under 35 U.S.C. 119(e) of U.S. Provisional Patent Application Ser. No. 60/521,565, filed May 25, 2004, which applications are incorporated herein by reference.

### TECHNICAL FIELD

Invention embodiments described herein relate to deodorant body wash products that cleanse and moisturize skin of a user through foaming action, as well as act as a deodorant. Embodiments also include methods of making the deodorant body wash, and methods of using the deodorant body wash.

### COPYRIGHT

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever. The following notice applies to any software and data as described below and in the drawings that form a part of this document: Copyright 2005, Coty, Inc. All Rights Reserved.

### BACKGROUND OF THE INVENTION

Personal care products have a variety of applications, including topical application to skin. The topical applications have acted to moisturize, cleanse, disinfect or to apply active agents to the skin. Typically, the topical applications have performed only one of these functions. One type of personal care product, a product that cleanses skin has, in some instances, used a structurant in order to produce a composition having a lamellar or spherulite phase.

It has been reported that formation of lamellar dispersion based compositions can only be accomplished with a limited, small group of surfactants. Surfactants not falling within this small group have been reported to crystallize out of solution when added to a composition containing a structurant, or to destabilize the composition or both. The WO 97/05857 patent application includes a table that describes performances of a collection of test surfactants in forming a lamellar phase when added to a composition containing a structurant. The data in the table stated that stearyl alcohol, glyceryl monostearate and cetyl alcohol did not form lamellar phases at room temperature. Instead, these materials crystallized out of solution or destabilized the lamellar phase formed by the composition.

Normal human beings produce about 0.5 to 1 liter of sweat daily. In cases of strain on the body and increased metabolism, human beings produce many times that amount. The course of the development of a human involves the formation of two types of sweat glands. From birth to puberty, a person has only ecrine sweat glands, small sweat glands, but the onset of puberty sees the formation of the apocrine sweat glands, large sweat glands, primarily in the area of the armpits and in the anal and genital regions. Only the apocrine sweat glands produce, in connection with skin bacteria which decompose the odorless sweat, sweat with the known unpleasant odors. The odor of sweat is person-specific and is pronounced to different degrees for each person. For most people, simple washing of skin achieves only a short-term improvement, meaning that often enough it is not possible to significantly reduce body odor without the use of deodorant active ingredients.

In order to achieve a deodorant effect, methods and antiperspirant formulations are used in combination. The use of antiperspirants that include aluminum and aluminum/zirconium salts, which prevent the production of sweat by blocking the openings of sweat glands has been known for a long time. According to recent findings, the reduced sweat production has no effect on the organism since the “cooling effect” largely takes place via the “sweating” of the ecrine glands. The inhibition of bacterial growth as a result of bacteriostats in the area of the skin zones covered with apocrine sweat glands is not acceptable and sometimes leads to severe irritations and allergic reactions.

Ethyl alcohol, which is present in many of the conventional deodorant products, also acts as bacteriocide. To mask the odor of sweat, fragrances or perfume substances are frequently present in the deodorant preparations. Some of these fragrances also have a bacteriostatic effect, but with many of the fragrances or perfumes, users have similar side-effects as imparted by the bacteriostats.

### DESCRIPTION

Embodiments of the invention described herein include formulation and system embodiments for cleansing and moisturizing skin and for acting as a deodorant, wherein the formulation and system embodiments include a stable, aqueous dispersion of cleansing and moisturizing agents that are structured within a stable, spheroidal network of finely divided cleansing and moisturizing particles. The stable, spheroidal network is capable of foaming due to flocculation in water and mechanical action by a consumer, which, in one embodiment, occurs in the shower. The spheroidal network also includes wetting agents and emulsifiers such as stearic acid, cetyl alcohol, glyceryl monostearate and stearyl alcohol, that are incorporated within the network. The wetting agents and emulsifiers are desirable because they aid in building viscosity of the formulation, and aid in producing a high yield value. Further, the wetting agents and emulsifiers aid in skin occlusiveness for increased moisturization. While specific types of wetting agents and emulsifiers are described herein, it is understood that embodiments of the invention described herein are not limited to the specific wetting agents and emulsifiers described.

Embodiments of the invention additionally include methods for making the formulation and system, as well as methods for adding wetting agents and emulsifiers to a structurant in a manner that prevents the wetting agents and emulsifiers from “salting out.” Furthermore, the wetting agents and emulsifiers such as stearic acid, cetyl alcohol, glyceryl monostearate and stearyl alcohol do not destabilize the activity of the structurant. Embodiments of the invention also include methods for using the formulation and system of the invention to make a deodorizing product.

Prior to embodiments of the invention described herein, it has not been thought possible to incorporate wetting agents and emulsifiers such as stearic acid, cetyl alcohol, glyceryl monostearate, and stearyl alcohol into a formulation with a structurant to make a spheroidal network. Embodiments of the invention described herein include stearic acid, cetyl alcohol, glyceryl monostearate and stearyl alcohol as well as a structurant to make the spheroidal network. It has surprisingly been found that the stearic acid, cetyl alcohol, glyceryl monostearate, and stearyl alcohol do not salt out of the network and do not destabilize the network but are incorporated into the network without introducing instability. To the contrary, it has unexpectedly been found that these wetting agents aid in building viscosity of the spheroidal network and aid in producing a high yield value. While stearic acid, cetyl alcohol, glyceryl monostearate, and stearyl alcohol are described, it is understood that other alcohols and waxes are suitable for use in the stable formulation embodiments of the invention.

It has also surprisingly been found that the deodorant embodiments described herein, which are applied to skin in the shower, perform in a manner comparable to a conventional deodorant product. It has not been heretofore thought possible to deodorize skin, long term, using a cleansing wash.

As used herein, the term “lamellar” refers to an ordered liquid crystalline phase having alternating surfactant bilayers and water layers.

The term “spheroidal network” as used herein refers to a lamellar phase that conforms to form a submicron spherical onion. Vesicles and liposomes are types of spheroidal networks.

The term “structurant” as used herein refers to a molecule that aids in the formation of a spheroidal network.

The term “lotion” refers to a cosmetic formulation applied to the skin.

The term “wash” refers to a cosmetic formulation that is applied to the skin and then is washed from the skin.

The spheroidal network included in invention embodiments described herein has a multilayer structure conformed to a submicron onion shape. Insoluble materials are dispersed throughout the onion shaped spheroidal network. For some embodiments, insoluble materials are enclosed within the interior layers of the spheroidal network as well as the outer layers. Soluble materials are similarly dispersed throughout the spheroidal network, within interior layers and outer layers.

A structurant is a component of cleansing-moisturizing wash embodiments of the invention. Composition embodiments of the invention that employ structurants have, in some embodiments, lamellar or spherulitic phases that are capable of suspending large particles within the phase while remaining pourable. Structurants are also used to prepare product embodiments of the invention that impart a soft feel that is pleasing to consumers. One type of structurant is an electrolyte-based structurant. Examples of electrolyte-based structurants usable in the formulation and system embodiments of the invention described herein are described in WO/0105932, assigned to Huntsman and U.S. Patent Publication 20030190302, assigned to Rhodia. While specific electrolyte-based structurants are described herein, it is believed that other electrolyte-based structurants are suitable for use in embodiments of the invention.

One cleansing-moisturizing wash embodiment of the invention has foaming functionality that aids in the cleansing functionality. This embodiment of the cleansing-moisturizing wash includes five phases. Ingredients in the five phases for one embodiment are shown in the table that follows. It is understood that this embodiment is presented as one example of the invention described herein and is not presented to limit embodiments of the invention.

| | % w/w |
|---|---|
| Phase A | |
| Deionized Water | 7.000 |
| Guar hydroxypropyltrimonium chloride | 0.500 |
| Phase B | |
| Glycerine 99% USP | 1.000 |
| Cyamposis Tetragonoloba (Guar) Gum | 0.300 |
| Structurant Blend | 33.000 |
| Phase C | |
| Sodium Laureth Sulfate | 5.000 |
| Phase D | |
| Deionized Water | 25.700 |
| EDTA disodium salt | 0.070 |
| Uvinul MS 40 Powder | 0.200 |
| Grape Seed Oil | 2.000 |
| Silicone 200/500 | 0.750 |
| Ethyl Hexyl Hydroxystearate | 1.000 |
| C12-15 Alkyl Ethyl Hexanoate | 0.750 |
| Stearic Acid Tri Press | 0.500 |
| Cetyl Alcohol | 0.500 |
| Glycerol Monostearate | 0.250 |
| Stearyl Alcohol | 0.500 |
| Petrolatum | 6.000 |
| Shea Butter | 3.000 |
| Farnesol | 0.200 |
| Triethyl Citrate | 2.400 |
| Phase E | |
| Fragrance | 1.250 |
| NaCl (Neat) | 5.000 |
| Citric Acid 20% Aq. Soln. | 1.500 |
| Phase E | |
| Phenonip | 1.000 |
| Timica Pearl White | 0.500 |

A first phase, phase A, of the wash includes a deionized water diluent, a cationic conditioning agent, guar hydroxypropyltrimonium chloride. Other cation conditioning polymers which are suitable for use in phase A, include Polyquatemium-4, Polyquatemium-6, Polyquatemium-7, Polyquaternium-10, Polyquaternium-11, Polyquaternium-16, Polyquarternium-24, and Polyquaternium-39. While specific quantities of ingredients are described in the table, it is understood that other concentration ranges may be suitable for use in formulation embodiments of the invention. The diluent range varies in accordance with other ingredients to reach a total concentration of 100 percent by weight. The cationic conditioning range may be from 0.001 to 1.000 percent by weight.

Phase B includes glycerine, the surfactant blend in a concentration of about 30 to 40 percent, and the cyamopsis tetragonoloba (guar) gum. In one embodiment, the surfactant blend is sodium lauroamphoacetate, sodium trideceth sulfate and cocamide MEA.

Phase D includes lotion ingredients such as grape seed oil, silicones, esters, wetting agents and emulsifiers such as stearic acid tri press, cetyl alcohol, glycerol monostearate, stearyl alcohol, sodium lauryl sulphate, fatty alcohol, ether sulfates, disodium-n-lauryl-β-imino dipropionate, polyoxyethylenized castor oil, or sorbitan monooleate, sorbitan monostearate, lecithin, polyoxyethylene stearate, alkyl phenol polyglycol ether, cetyltrimethyl ammonium chloride, or mono-/dialkylpolyglycol ether-orthophosphorus acid-mono-ethanolamine salts, petrolatum, shea butter, Famesol, Triethylcitrate, and a chelating agent, EDTA disodium salt and other non-toxic salts. The chelating agent range may be from 0.001 to 0.250 by weight. Other ingredients are suitable for use in phase D to formulate a lotion.

Phase E includes sodium chloride and citric acid. The concentration range for use of sodium chloride is from 1.000 to 6.000 percent by weight. The concentration range for use of citric acid is from 0.001 to 3.000 percent by weight. If required, the pH is adjustable with sodium hydroxide or any other pH adjusting electrolyte, also within a range of 0.001 to 3.000 percent.

Phase F includes a preservative, Phenonip. Other preservatives suitable for use include DMDM Hydantoin, phenoxyethanol, parabens, chlorophenesin, benzyl alcohol, chlorhexidine gluconate, an ethyl alcohol containing pentylene glycol and a sodium methylparaben mixture in the proportions 47/47/6, a pentylene glycol and sodium methylparaben mixture, methylchloroisothiazolinone, methylisothiazolinone, and mixtures thereof in a concentration range of 0.001 to 1.000 percent by weight, based upon proven efficacy per formula embodiment.

In addition to the above noted compounds, various other ingredients can optionally be utilized in the stable composition of embodiments of the present invention such as fragrances, perfumes, preservatives, disinfectants, antioxidants, antiredeposition agents, carriers, chelating and sequestering agents, dyes and pigments, quaternary conditioners, cationic conditioning polymers such as, polyquatemium-4, polyquatemium-6, polyquaternium-7, polyquaternium-10; polyquatemium-11, polyquatemium-16, polyquarternium-24, and polyquaternium-39, corrosion inhibitors, hydrotropes, coupling agents, defoamers, builders, dispersants, emollients, extracts, vitamins, enzymes, foam boosters, flocculants, whitening agents, fixative polymers such as PVP, humectants, opacifiers, plasticizers, powders, solubilizers, solvents, waxes, UV absorbers/UV light stabilizers, hydrolyzed proteins, keratin, collagens, and the like.

Specific deodorants usable in deodorant wash embodiments include the following acidic aluminum and/or aluminum/zirconium salts that can be stably incorporated into the emulsions. Concentrations of 0.001% to 40% by weight, and, in particular 0.001% to 25% by weight, of aluminum chlorohydrate and/or aluminum/zirconium chlorohydrate can be stably incorporated into the emulsions. These concentration ranges refer to the active contents of the antiperspirant complexes, such as aluminum compounds and anhydrous complexes of aluminum compounds, as well as aluminum/zirconium compounds and anhydrous and buffer-free complexes of aluminum/zirconium compounds. One buffer usable herein includes glycine.

Antiperspirant actives suitable for use herein include astringent active salts, including in particular aluminium, zirconium and mixed aluminium/zirconium salts, including both inorganic salts, salts with organic anions and complexes. Astringent salts include aluminium, zirconium and aluminium/zirconium halides and halohydrate salts, such as chlorohydrates.

Aluminium halohydrates are usually defined by the general formula, Al₂(OH)ₓQ_{y}wH₂O, in which Q represents chlorine, bromine or iodine, x is variable from 2 to 5 and x+y=6 while wH₂O represents a variable amount of hydration.

Zirconium actives are represented by the empirical general formula: ZrO(OH)²ⁿ_{-nz}B_{z}. wH₂O in which z is a variable in the range of from 0.9 to 2.0 so that the value 2n-nz is zero or positive, n is the valency of B, and B is selected from the group consisting of chloride, other halide, sulphamate, sulphate and mixtures thereof. Possible hydration to a variable extent is represented by wH 20. For some embodiments, B represents chloride and the variable z lies in the range from 1.5 to 1.87. In practice, such zirconium salts are usually not employed by themselves, but as a component of a combined aluminium and zirconium-based antiperspirant.

The above aluminium and zirconium salts may have coordinated and/or bound water in various quantities and/or may be present as polymeric species, mixtures or complexes. In particular, zirconium hydroxy salts often represent a range of salts having various amounts of the hydroxy group. Zirconium aluminium chlorohydrate are usable for some embodiments.

Antiperspirant complexes based on the above-mentioned astringent aluminium and/or zirconium salts are employed for some embodiments. The complex often employs a compound with a carboxylate group, and advantageously this is an amino acid. Examples of suitable amino acids include dl-tryptophan, dl-β-phenylalanine, dl-valine, dl-methionine and β-alanine, and preferably glycine which has the formula CH₂(NH₂)COOH.

Some embodiments employ complexes of a combination of aluminium halohydrates and zirconium chlorohydrates together with amino acids such as glycine. Certain of those Al/Zr complexes are commonly called ZAG in the literature. ZAG actives generally contain aluminium, zirconium and chloride with an Al/Zr ratio in a range from 2 to 10, especially 2 to 6, an Al/Cl ratio from 2.1 to 0.9 and a variable amount of glycine. Actives of this type are available from Westwood, Summit and Reheis.

Some antiperspirant actives are produced in the form of dense particles, that is to say are free from voids, or hollow particles that have been milled. It is highly desirable to employ such particles in the context of forming formulations that are translucent when extruded through a narrow aperture and that can be used in opaque formulations. Other antiperspirant actives that are produced and remain in the form of hollow spheres are particularly suitable for opaque formulations.

The proportion of solid antiperspirant salt in a composition normally includes the weight of any water of hydration and any complexing agent that may also be present in the solid active. Such hydrated water does not render the formulation hydrous.

The particle size of the antiperspirant salts often falls within the range of 0.1 to 200 μm with a mean particle size often from 3 to 20 μm. Both larger and smaller mean particle sizes can also be contemplated such as from 20 to 50 μm or 0.1 to 3 μm.

Antiperspirant agents that are usable in formulation embodiments described herein include but are not limited to aluminum salts (of the empirical formula [Al₂(OH)ₘClₙ], where m+n=6); aluminum salts, such as aluminum chloride AlCl₃, aluminum sulfate Al₂(SO₄)₃; aluminum chlorohydrate [Al₂(OH)₅Cl]×H₂O standard Al complexes: Locron L (Clariant), Chlorhydrol (Reheis), ACH-303 (Summit), Aloxicoll L (Giulini). activated Al complexes: Reach 501 (Reheis), MCH-324 (Summit); aluminum sesquichlorohydrate [Al₂(OH)_{4.5}Cl_{1.5}]×H₂O standard Al complexes: aluminum sesquichlorohydrate (Reheis), ACH-308 (Summit), Aloxicoll 31 L (Giulini) activated Al complexes: Reach 301 (Reheis); aluminum dichlorohydrate [Al₂(OH)₄Cl₂]×H₂O; aluminum/zirconium salts: aluminum/zirconium trichlorohydrex glycine [Al₄Zr(OH)₁₃Cl₃]×H₂O×Gly standard Al/Zr complexes: Rezal 33GP (Reheis), AZG-7164 (Summit), Zirkonal P3G (Giulini) activated Al/Zr complexes: Reach AZZ 902 (Reheis), AAZG-7160 (Summit), Zirkonal AP3G (Giulini); aluminum/zirconium tetrachlorohydrex glycine [Al₄Zr(OH)₁₂Cl₄]×H₂O×Gly standard Al/Zr complexes: Rezal 36G (Reheis), AZG-368 (Summit), Zirkonal L435G (Giulini) activated Al/Zr complexes: Reach AZP 855 (Reheis), AAZG-6313-15 (Summit), Zirkonal AP4G (Giulini); aluminum/zirconium pentachlorohydrex glycine [Al₈Zr(OH)₂₃Cl₅]ₓH₂O×Gly standard Al/Zr complexes: Rezal 67 (Reheis), Zirkonal L540 (Giulini) activated Al/Zr complexes: Reach AZN 885 (Reheis); aluminum/zirconium octachlorohydrex glycine [Al₈Zr(OH)₂₀Cl₈]×H₂O×Gly.

Use of the antiperspirant agents from the raw material classes of aluminum and aluminum/zirconium salts is not limited to the standard commercial mainly aqueous solutions, such as, for example, Locron L (Clariant). Commercial anhydrous powders of the same raw materials, such as, for example, Locron P (Clariant) are also useful.

The use AT-salt suspensions in which aluminum and aluminum/zirconium salts present in powder form are supplied dispersed in various oils are usable for some embodiments.

Furthermore, it may be advantageous to use special aluminum and aluminum/zirconium salts which are supplied for improving the solubility as glycol complexes.

Further advantageous antiperspirant agents are based, instead of on aluminum or zirconium, on other metals, such as, for example, beryllium, titanium and hafnium.

In this connection, the list of antiperspirant agents which are usable should, however, not be limited to metal-containing raw materials, but compounds which comprise nonmetals, such as boron, and those which are classed as being in the field of organic chemistry, such as, for example, anticholergics, are also advantageous. Advantageous in this sense are also polymers which may either contain metals or be metal-free.

The effect arising in numerous preparations of a visible white residue remaining on the skin following application of the preparation is usually perceived by the user as being undesirable. In anhydrous preparations, the use of propoxylated alcohols has proven useful for concealing this phenomenon. The addition of propoxylated alcohols having 10 to 20 propyloxy units and 2 to 10 carbon atoms in the alkyl chain, in one particular embodiment. PPG-14 butyl ether, is usable to conceal the appearance of such white residues.

Deodorants are advantageously added to preparation embodiments described herein.

A use of antimicrobial substances in cosmetic deodorants reduces the bacterial flora on the skin. In this regard, only the odor-causing microorganisms should be effectively reduced. The flow of sweat itself is not influenced by this, and microbial decomposition of the sweat is merely temporarily stopped. The combination of astringents with antimicrobially effective substances in a composition is also usable.

Deodorants usable in invention embodiments described herein include, for example, odor concealers, such as perfume constituents, odor absorbers such as sheet silicates, in particular, montmorillonite, kaolinite, illite, beidellite, nontronite, saponite, hectorite, bentonite, smectite, and also, zinc salts of ricinoleic acid. Antimicrobial agents are likewise suitable for incorporation into emulsions according to embodiments described herein. Advantageous substances are, for example, 2,4,4′-trichloro-2′-hydroxydiphenyl ether (Irgasan), 1,6-di(4-chlorophenylbiguanido)hexane (chlorhexidine), 3,4,4′-trichlorocarbanilide, quaternary ammonium compounds, oil of cloves, mint oil, thyme oil, triethyl citrate, ethylhexyl glycerine, polyglycerol caprylate, and famesol (3,7,11-trimethyl-2,6,10-dodecatrien-1-ol). Sodium hydrogencarbonate is also usable for some embodiments.

Other deodorant constituents include deoperfumes, and/or microbicides, including particularly bactericides, such as chlorinated aromatics, including biguanide derivatives, such as materials triclosan (Irgasan DP300®), chlorhexidine and Tricloban®. A yet another class includes biguanide salts such as are available under the trade mark Cosmosil®.

A yet further class of antimicrobial which can advantageously be employed herein comprises transition metal chelators, such as amino acids or salts thereof, which chelators have affinity for iron (III), and a binding constant for iron (III) of greater than 10¹⁰, or, for optimum performance, greater than 10²⁶. The iron (III) binding constant referred to above is the absolute stability constant for the chelator-iron (III) complex. One especially preferred chelator is DTPA (diethylene triamine pentaacetic acid) and salts thereof. Such antimicrobials suppress microbial regrowth when used in amounts ranging from 0.35 to 2% by weight.

Active ingredients or active ingredient combinations which are usable in the emulsions according to embodiments the invention are not intended to be limiting. The amount of deodorants (one or more compounds) in the preparations is for some embodiments, 0.01 to 10% by weight, particularly 0.05 to 5% by weight, in particular 0.1 to 1% by weight, based on the total weight of the preparation.

Other optional ingredients can be incorporated to the extent that they are miscible with the carrier fluids. They include skin benefit agents such as glycerol as mentioned previously herein, and allantoin or lipids, for example, in an amount of up to 5%; oil-soluble colorants; skin cooling agents such as menthol and menthol derivatives, often in an amount of up to 2%, all of these percentages being by weight of the formulation. A commonly employed and highly desired ingredient is a perfume, which is normally present at a concentration of from 0 to 4% and in many formulations from 0.25 to 2% by weight thereof.

In the formulation embodiment of the table described above, phase A was prepared by mixing the guar hydroxypropyltrimonium chloride and deionized water until the guar hydroxypropyltrimonium chloride was thoroughly blended to form a uniform phase A mixture.

To make phase B, the Cyamopsis Tetragonoloba (Guar) Gum and glycerine are blended until the Cyamopsis Tetragonoloba (Guar) Gum was wetted and was completely dispersed to make a slurry. The glycerine/Cyamopsis Tetragonoloba (Guar) Gum slurry was added to the surfactant blend and was mixed until a uniform blend was achieved. The phase B uniform blend was then added to the phase A mixture to form a combined phase A and B mixture.

The combined phase A and B mixture is mixed until uniform. The uniform mixture was then heated to a temperature within a range of 40° C. to 70° C. Phase C was added to the combined phase A and B mixture to make a phase A, B and C mixture.

To make phase D, the following ingredients were blended: EDTA disodium salt, grape seed oil, silicone 200/500, EthylHexyl Hydrostearate, C12-15 Alkyl EthylHexanoate, stearic acid tri pres, cetyl alcohol, glycerol monostearate, stearyl alcohol, petrolatum, shea butter, famseol, triethyl citrate and deionized water. The phase D mixture was heated until all ingredients were liquid. When all ingredients were liquid, rendering phase D liquid, phase D was high energy mixed at moderate speed. The moderate speed mixing was continued until the mixture was ready to add to the combined mixture of phases A, B and C, also called the main batch.

Phase D was then added to make a combined blend of phase A, B, C and D. The mixture of phase A, phase B, phase C and phase D was mixed until a homogeneous mixture was achieved. The mixture was cooled to room temperature at a rate of 1° C. for every 10 minutes. The ingredients of phases E and F were added, one at a time, until a homogeneous mixture that had all of the desired attributes of the cleansing foaming lotion embodiment of the invention were achieved.

The cleansing foaming lotion embodiments of the invention described herein are viscous liquids, having a viscosity of about 100,000 cPs and a pH of 5.5 to 6.5. The wash embodiments, are, for some embodiments, colored or fragranced. Some embodiments of the cleansing foaming lotion may moisturize skin for at least about 24 hours from application.

One lotion embodiment of the invention foams and cleanses for use in the shower and may moisturize skin for at least about 24 hours after showering. With this embodiment, a user need use only a product of the invention to both cleanse and moisturize. A separate cleanser and moisturizer are not required. That the invention described herein is capable of both cleansing and moisturizing is unexpected because cleansing skin removes fats, oils and lipids from the skin. Cleansing typically leaves skin dehydrated. It is then unexpected that a single product is capable of both cleansing and moisturizing.

One additional attribute of the wash of the invention described herein is that the cleansing moisturizing wash moisturizes skin without leaving a heavy oil feel on the skin. This heavy moisturized feel typically occurs when a moisturizer is separately applied to skin. The wash described herein not only saves a user time and money in not having to apply two separate products but eliminates the heavy oil feel while effectively moisturizing.

The cleansing foaming wash of the invention described herein differs from a traditional wash in that the traditional wash does not include a spherulite state. This spherulite state is also known as an “onion phase” state. The spherulite state is a stable high energy state. The spherulites within the wash make it possible to add lotion ingredients to a cleansing and foaming product and to maintain all of the properties of both the body wash, which cleanses and the lotion, which moisturizes.

The cleansing foaming wash with a lotion is a structured liquid formulation that includes water soluble, water dispersible, water insoluble and water indispersible ingredients without an adverse impact, such as “salting out” and incipient instability. The cleansing portion of the formulation also includes adjuvants and solubilizers that aid in creating a product with a pre-selected viscosity or foaming potential. The formulation of the invention described herein produces a finished product that has improved stability as compared to conventional washes because of materials in the formulation, that add stability, and that cannot be added to a conventional wash. These materials include wetting agents and emulsifiers such as stearic acid, cetyl alcohol, glyceryl monostearate and stearyl alcohol.

A method for making a formulation embodiment of the invention that includes waxes and alcohols such as stearic acid, cetyl alcohol, glyceryl monostearate and stearyl alcohol, or other wax or alcohol-based surfactant, includes the steps described herein. One embodiment of phase D, such as is described above, was prepared by adding oils, silicones, esters and any other insoluble ingredients to the alcohols and waxes. About 10 percent deionized water was also added.

Phase D was heated to about 70° C. to melt the alcohols and waxes. When all of the alcohols and waxes were melted, phase D was emulsified and homogenized using, in one embodiment, a homo-mixer at moderate speed. Phase D was mixed for a minimum of five minutes. Moderate sidesweep mixing was continued until the phase D was ready for addition to the phase A, B and C mixture. Phase D was then added to the phase A, B and C mixture when the A, B, and C mixture was mixed homogeneously. An addition of sodium chloride and/or citric acid and/or fragrance drove the formula to a spherulite phase. It was observed that the spherulite phase was stable with no salting out.

One test for determining whether the formulation is in a spherulite phase includes taking a 100 gram formulation sample of the formulation and adding 1 to 2 percent neat salt. If, at 24 hours the viscosity has increased above the initial viscosity of the batch, the formulation is not in the spherulite phase. This result indicates that the formulation requires more oils or salt in order to form a spherulite phase. A second test includes preparing a sample of about 100 grams and adding several beads to the sample. The sample is held at a temperature of about 50° C. for several days. If the beads are still suspended, the sample has a spherulite phase and is stable.

Embodiments of the formulation of the invention described herein have use in shaving and moisturizing, shampoos and conditioners, in addition to washes and moisturizers. Formulation embodiments of the invention described herein have a wide number of other applications such as personal care applications, home care applications, industrial and institutional applications, pharmaceutical applications, textile compounds, and the like.

Examples of various personal care applications include products such as the following: shampoos, for example, baby shampoos; conditioning shampoos; bodifying shampoos; moisturizing shampoos; temporary hair color shampoos; 3-in-1 shampoos; anti-dandruff shampoos; hair color maintenance shampoos; acid (neutralizing) shampoos; and salicylic acid shampoos.

Skin and body cleansers, for example, moisturizing body washes; antibacterial body washes; deodorizing body washes; bath gels; shower gels; hand soaps; bar soaps; body scrubs; bubble baths; facial scrubs; foot scrubs; creams and lotions, for example, alpha-hydroxy acid lotions and creams; beta-hydroxy acid creams and lotions; skin whiteners; self tanning lotions; sunscreen lotions; barrier lotions; moisturizers; hair styling creams; vitamin C creams; liquid talc products and antibacterial lotions; and other moisturizing lotions and creams.

One specific embodiment is a formulation for a 24 hour deodorant body wash. Three formulations are shown as follows:

24 hour Deodorant Body Wash with Lotion

| | −1 | −2 | −3 |
|---|---|---|---|
| | % w/w | % w/w | % w/w |
| Phase A | | | |
| DI Water | 22.080 | 21.880 | 24.380 |
| Jaguar C-17 | 0.500 | 0.500 | 0.500 |
| Phase B | | | |
| Glycerine 99% USP | 1.000 | 1.000 | 1.000 |
| Jaguar S | 0.300 | 0.300 | 0.300 |
| Miracare SLB-365 | 33.000 | 33.000 | 33.000 |
| Phase C | | | |
| Sodium Laureth Sulfate | 5.000 | 5.000 | 5.000 |
| Phase D | | | |
| Uvinul MS 40 Powder | 0.200 | 0.200 | 0.200 |
| Trilon BD | 0.070 | 0.070 | 0.070 |
| Grape Seed Oil | 2.000 | 2.000 | 2.000 |
| Silicone 200/500 | 0.750 | 0.750 | 0.750 |
| Wickenol 171 | 1.000 | 1.000 | 1.000 |
| Hetester FAO | 0.750 | 0.750 | 0.750 |
| Emersol 7036 (Stearic Acid) | 0.500 | 0.500 | 0.500 |
| Cetyl Alcohol | 0.500 | 0.500 | 0.500 |
| Glyceryl Monostearate | 0.250 | 0.250 | 0.250 |
| Steryl Alcohol | 0.500 | 0.500 | 0.500 |
| Petrolatum | 6.000 | 6.000 | 6.000 |
| Shea Butter | 3.000 | 3.000 | 3.000 |
| Farnesol | 0.200 | 0.000 | 0.000 |
| TriethylCitrate | 2.400 | 2.400 | 0.000 |
| EthylHexylGlycerine | 0.000 | 0.400 | 0.000 |
| Triclosan | 0.000 | 0.000 | 0.300 |
| DI Water | 10.000 | 10.000 | 10.000 |
| Phase E | | | |
| EO . . . | 2.000 | 2.000 | 2.000 |
| CaCl (Neat) | 5.000 | 5.000 | 5.000 |
| Citric Acid 20% Aq. Soln | 1.500 | 1.500 | 1.500 |
| Phase E | | | |
| Phenonip | 1.000 | 1.000 | 1.000 |
| Timica Pearl White | 0.500 | 0.500 | 0.500 |

When formulations such as the deodorant formulation above were applied to skin, it was found that residual deodorizing ingredients were retained on the skin for at least about 24 hours after application to skin.

Examples of other applications include home care applications that include products such as home care and industrial and institutional applications, such as laundry detergents; dishwashing detergents (automatic and manual); hard surface cleaners; hand soaps, cleaners and sanitizers; polishes (shoe, furniture, metal, etc.); automotive waxes, polishes, protectants, and cleaners, and the like.

Examples of pharmaceutical applications include topical formulations in the form of creams, lotions, ointments, or gels, wherein the formulation may be used as a carrier for the pharmaceutically active material, or as a carrier for a skin penetration enhancer, or as a carrier for a phase having an aesthetic effect, or present to enhance the solubility or bioavailability of the pharmaceutically active material.

These formulations may be administered or applied to either human or veterinary conditions for the full breadth of indications treatable by pharmaceutical means, such as fever, irritation, dermatitis, rash; viral, fungal, or bacterial infection; organic disease; etc. The pharmaceutically active agents could have any appropriate function for treatment of the condition, and can be a mixture of one or more pharmaceutically active materials, such as emetics, antiemetics, febrifuge, fungicide, biocide, bactericide, antibiotic, antipyretic, NSAID, emollient, analgesics, antineoplastics, cardiovascular agents, enzymes, proteins, hormones, steroids, antipruritics, antirheumatic agents, biologicals, cough and cold treatments, dandruff products, muscle relaxants, psychotherapeutic agents, skin and mucous membrane agents, skin care products, vaginal preparations, wound care agents, and other appropriate classes of pharmaceutically active agents capable of appropriate administration via dosage form.

The formulation embodiments may be packaged in a pressurized container or unpressurized container. The formulation may be applied to wipes, swabs or other flexible substrates.

The formulation embodiments may include variegation, and suspended solids that impart color. The formulation embodiments may be made into a wide variety of product types that include, but are not limited to, lotions, creams, gels, sticks, sprays, ointments, cleansing liquid washes, solid bars, shampoos, pastes, foams, powders, mousses, shaving creams, wipes, patches, nail lacquers, wound dressing, adhesive bandages, hydrogels, and films. Make-up, such as foundations, mascaras, and lipsticks also form suitable compositions. These product embodiments may further comprise several additional types of cosmetically acceptable topical carriers including, but not limited to solutions, emulsions (e.g., microemulsions and nanoemulsions), gels, solids and liposomes.

While certain embodiments of the present invention have been described and specifically exemplified above, it is not intended that the invention be limited to such embodiments. Various modifications may be made thereto without departing from the scope and spirit of the present invention, as set forth in the following claims.

1. A deodorant formulation comprising: a structurant and an emulsion comprising a homogenized mixture of wax and alcohol components, at least one of which is a surfactant, and a deodorant wherein the formulation comprises a stable lamellar or spherulite phase.

2. The formulation of claim 1 wherein the wax is melted.

3. The formulation of claim 1 wherein the structurant comprises an electrolyte.

4. The formulation of claim 1, wherein the emulsion further comprises grape seed oil.

5. The formulation of claim 1, wherein the emulsion further comprises one or more silicone.

6. The formulation of claim 1, wherein the emulsion further comprises one or more ester.

7. The formulation of claim 1, wherein the emulsion further comprises water.

8. The formulation of claim 1, wherein the wax and alcohol components comprise one or more of stearic acid, cetyl alcohol, glyceryl monostearate, and stearyl alcohol.

9. The formulation of claim 1, further comprising water.

10. The formulation of claim 1, further comprising a fragrance.

11. The formulation of claim 1 further comprising one or more ingredients that moisturize skin.

12. The formulation of claim 10 wherein the ingredients that moisturize skin comprise one or more of grape seed oil, silicone, and esters.

13. The formulation of claim 1 further comprising one or more ingredients that cleanse skin.

14. The formulation of claim 1 further comprising one or more ingredients that cleanse and moisturize skin.

15. A wipe comprising the deodorant formulation of claim 1.

16. The formulation of claim 1 comprising five phases.

17. The formulation of claim 9 comprising a lotion phase.

18. The formulation of claim 10 wherein the lotion phase comprises one or more of stearic acid, cetyl alcohol, glyceryl monostearate, and stearyl alcohol.

19. A method for deodorizing a human body comprising applying the formulation of claim 1 to the body in a shower.

20. A method for deodorizing and cleansing a human body comprising applying the formulation of claim 1 to the body in a shower.
